(19) United States
(12) Patent Application Publication
(10) Pub. No.: US 2002/0006963 A1
(43) Pub. Date: Jan. 17, 2002

(54) Method of using and compositions comprising (-) sibutramine optionally in combination with other pharmacologically active compounds

(76) Inventors: James W. Young, Palo Alto, CA (US); Thomas P. Jerussi, Framingham, MA (US)

Correspondence Address: PENNIE & EDMONDS LLP, 1667 K STREET NW, SUITE 1000, WASHINGTON, DC, 20006

(21) Appl. No.: 09/770,665

(22) Filed: Jan. 29, 2001

Related U.S. Application Data
(63) Continuation-in-part of application No. 09/721,669, filed on Nov. 27, 2000.
Application No. 09/721,669 is a continuation of application No. 08/461,608, filed on Jun. 5, 1995, now abandoned.
Application No. 08/461,608 is a continuation of application No. 07/903,040, filed on Jun. 23, 1992, now abandoned.

(51) Int. Cl. A61K 31/135; A61K 31/40; A61K 31/724; A61K 31/496; A61K 31/497

(52) U.S. Cl. 514/646; 514/258; 514/58; 514/252.16; 514/409; 514/255.06

(57) ABSTRACT

This invention encompasses methods for the treatment and prevention of disorders that include, but are not limited to, eating disorders; weight gain; obesity; irritable bowel syndrome; obsessive-compulsive disorders; platelet adhesion; apnea; affective disorders such as attention deficit disorders, depression, and anxiety; male and female sexual function disorders; restless leg syndrome; osteoarthritis; substance abuse including nicotine and cocaine addiction; narcolepsy; pain such as neuropathic pain, diabetic neuropathy, and chronic pain; migraines; cerebral function disorders; chronic disorders such as premenstrual syndrome; and incontinence.

The invention further encompasses pharmaceutical compositions and dosage forms which comprise optically pure (−) sibutramine, optionally in combination with a phosphodiesterase inhibitor or a lipase inhibitor.

This is a continuation-in-part of U.S. patent application 09/721,669, filed Nov. 27, 2000, which is a continuation of U.S. patent application 08/461,608, both of which are incorporated herein by reference in their entireties.

### 1. FIELD OF THE INVENTION

This invention is directed to methods and compositions for the treatment or prevention of conditions using optically pure (−) sibutramine, optionally in combination with other pharmacologically active compounds.

### 2. BACKGROUND OF THE INVENTION

2.1. Sibutramine

Sibutranmine, chemically named [N-1-[1-(4-chlorophenyl)cyclobutyl]-3-methylbutyl]-N,N-dimethylamine, is a neuronal monoamine reuptake inhibitor which was originally disclosed in U.S. Pat. Nos. 4,746,680 and 4,806,570. Sibutramine inhibits the reuptake of norepinephrine and, to a lesser extent, serotonin and dopamine. See, e.g., Buckett et al., Prog. Neuro-psychopharm. & Biol. Psychiat., 12:575-584, 1988; King et al., J. Clin. Pharm., 26:607-611 (1989).

Racemic sibutramine is sold as a hydrochloride monohydrate under the tradename MERIDIA®, and is indicated for the treatment of obesity. Physician 's Desk Reference® 1494-1498 (53^{rd} ed., 1999).

Sibutramine appears to have been extensively studied, and reportedly could be used in the treatment of a variety of disorders. For example, U.S. Pat. Nos. 4,552,828, 4,746,680, 4,806,570, and 4,929,629 disclose methods of treating depression using racemic sibutramine, and U.S. Pat. Nos. 4,871,774 and 4,939,175 disclose methods of treating Parkinson's disease and senile dementia, respectively, using racemic sibutramine.

While racemic sibutramine reportedly can be used in the treatment of a variety of diseases and conditions, it unfortunately has a number of adverse effects. Adverse effects associated with racemic sibutramine include, but are not limited to, significant increases in supine and standing heart rate, including tachycardia, increased blood pressure (hypertension), increased psychomotor activity, dry mouth, dental caries, constipation, hypohidrosis, blurred or blurry vision, tension, mydriasis, seizures, formation of gallstones, renal/hepatic dysfunction, fevers, arthritis, agitation, leg cramps, hypertonia, abnormal thinking, bronchitis, dyspnea, pruritus, amblyopia, menstrual disorder, ecchymosis/bleeding disorders, interstitial nephritis, and nervousness. These adverse effects may significantly limit the dose level, frequency, and duration of drug therapy.

2.2. Affective, Cerebral Function, and Other Disorders

This invention concerns, in part, methods of treating and preventing a variety of different diseases and conditions in patients. One is mania, which, like depression, is characterized by changes in mood as the primary symptom. Either of these two extremes of mood may be accompanied by psychosis with disordered thought and delusional perceptions. Psychosis may have, as a secondary symptom, a change in mood, and it is this overlap with depression that causes much confusion in diagnosis. Severe mood changes without psychosis frequently occur in depression and are often accompanied by anxiety.

Other disorders are affective disorders, which are characterized primarily by changes in mood. Major depression is the most common of the significant mental illnesses; it must be distinguished clinically from periods of normal grief, sadness, disappointment, and the related dysphoria or demoralization frequently associated with medical illness. Depression is characterized by feelings of intense sadness, despair, mental slowing, loss of concentration, pessimistic worry, agitation, and self-deprecation. Physical changes can also occur, including insonmia, anorexia, weight loss, decreased energy, loss of libido, and disruption of hormonal circadian rhythms. Often the condition responds to tricyclic or related antidepressant drugs or monoamine oxidase inhibitors.

This invention also concerns the treatment and prevention of dementia, which includes Alzheimer's-type dementia, is produced by a degenerative process involving a loss of cerebral cortical cells; memory loss is a prominent symptom. Dementia is a syndrome of progressive and irreversible dysfunction, presumably caused by cerebral neuropathologic changes and cell loss. The condition is considered to be dominated by cognitive difficulties; depression, paranoia, anxiety, and other psychologic symptoms may also be predominant. In sum, the common clinical profile is one of slow disintegration of both personality and intellect caused by impaired insight and judgment and by the loss of affect. Dementia is usually insidious, slowly progressive, and usually untreatable. However, in depressed, demented individuals, some antidepressants can significantly improve total function.

Alzheimer's type dementia may also be treated by antidepressant therapy. Alzheimer's type dementia (ATD) is a particularly devastating type dementia which affects 30% of humans over 80 years of age (See Evans et al., J.A.M.A. 262: 2551-2556, 1989). ATD is a neurodegenerative disease characterized by gradual cognitive impairment. The etiology and pathogenesis of this dementia is associated histopathologically with amyloid plaques, neurofibrillary tangles and loss of neuronal mass primarily in the brain's temporal lobe and neocortex. All of the above mentioned conditions may occur as a result of cerebral function disorders or cerebrovascular disease, and as such, racemic sibutramine may provide treatment and relief from ATD.

Other disorders of concern are cerebral function disorders, which have a complex etiology. Among their causes are cerebrovascular diseases such as cerebral infarction, cerebral bleeding, cerebral arteriosclerosis, cerebral venous thrombosis, and head injuries and the like. Cerebral function disorders produce a variety of symptoms as secondary diseases, for example, disturbances of consciousness, coma, lowering of attention, amnestic syndrome, senile dementia, speech disorder and the like.

Another disease of the central nervous system is Parkinson's disease, which is a chronic, progressive central nervous system disorder that usually appears insidiously in the later decades of life. The disease produces a slowly increasing disability in purposeful movement. It is characterized by the major clinical features of tremor, bradykinesia, rigidity, and a disturbance of posture. Patients often have an accompanying dementia. In idiopathic parkinsonism, there is usually a loss of cells in the substantia nigra, locus ceruleus, and other pigmented neurons of the brain, and a decrease of dopamine content in nerve axon terminals of cells projecting from the substantia nigra. The understanding that Parkinson's disease is a syndrome of dopamine deficiency resulted from a series of basic and clinical observations.

This invention is further directed to the treatment and prevention of obesity. Obesity is characterized by an accumulation of body fat, to the extent that body weight is 20 percent greater than standard. The importance of the condition is in the number of medical complications to which obese individuals are subject. While the etiology of obesity is simple and relates to consuming more calories than are expended, many factors contribute to the condition.

The prognosis for obesity is poor; it is a chronic condition that is resistant to treatment and prone to relapse. Caloric reduction through diet, increased physical activity, radical surgical treatment, and medication are considered treatments that may be employed in individual cases. Drug treatment of obesity is often governed by restrictive governmental regulation, and weight gain following this treatment modality is often greater than with other treatments.

### 3. SUMMARY OF THE INVENTION

This invention is directed, in part, to pharmaceutical compositions and dosage forms that comprise racemic sibutramine, or a pharmaceutically acceptable salt, solvate, hydrate, clathrate, or prodrug thereof, and a phosphodiesterase inhibitor. The invention further encompasses pharmaceutical compositions that comprise optically pure (−) sibutramine (i.e., (−) sibutramine substantially free of (+) sibutramine), or a pharmaceutically acceptable salt, solvate, hydrate, clathrate, or prodrug thereof, optionally in combination with a phosphodiesterase inhibitor.

The invention also relates to methods of treating or preventing a variety of diseases and conditions in patients (e.g., mammals such as humans), which comprise the administration of therapeutically or prophylactically effective amounts of racemic sibutramine and a phosphodiesterase inhibitor. Other methods of the invention comprise the administration of optically pure (−) sibutramine, or a pharmaceutically acceptable salt, solvate, hydrate, clathrate, or prodrug thereof, and the optional administration of a therapeutically or prophylactically effective amount of a phosphodiesterase inhibitor. Preferred methods of the invention that comprise the administration of optically pure (−) sibutramine avoid adverse effects associated with racemic sibutramine.

This invention encompasses methods for the treatment and prevention of disorders that include, but are not limited to, eating disorders; weight gain; obesity; irritable bowel syndrome; obsessive-compulsive disorders; platelet adhesion; apnea; affective disorders such as attention deficit disorders, depression, and anxiety; male and female sexual function disorders; restless leg syndrome; osteoarthritis; substance abuse including nicotine and cocaine addiction; narcolepsy; pain such as neuropathic pain, diabetic neuropathy, and chronic pain; migraines; cerebral function disorders; chronic disorders such as premenstrual syndrome; and incontinence.

3.1. Definitions

As used herein, the term “prodrug” means a derivative of a compound that can hydrolyze, oxidize, or otherwise react under biological conditions (in vitro or in vivo) to provide the compound. Examples of prodrugs include, but are not limited to, derivatives of (−) sibutramine that comprise biohydrolyzable moieties such as biohydrolyzable amides, biohydrolyzable esters, biohydrolyzable carbamates, biohydrolyzable carbonates, biobydrolyzable ureides, and biohydrolyzable phosphates. As used herein, prodrugs of optically pure (−) sibutramine do not include racemic sibutramine.

As used herein, the terms “biohydrolyzable carbamate,” “biohydrolyzable carbonate,” “biohydrolyzable ureide,” “biohydrolyzable phosphate” mean a carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “biohydrolyzable ester” means an ester of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein, the term “biohydrolyzable amide” means an amide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable amides include, but are not limited to, lower alkyl amides, α-amino acid amides, alkoxyacyl amides, and alkylaminoalkylcarbonyl amides.

As used herein, the term “biohydrolyzable ureide” means a ureide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound.

As used herein, the term “biohydrolyzable phosphate” means a phosphate of a compound that either: 1) does not interfere with the biological activity of the compound but can confer upon that compound advantageous properties in vivo, such as uptake, duration of action, or onset of action; or 2) is biologically inactive but is converted in vivo to the biologically active compound.

As used herein, the term “pharmaceutically acceptable salt” refers to a salt prepared from a pharmaceutically acceptable non-toxic inorganic or organic acid. Inorganic acids include, but are not limited to, hydrochloric, hydrobromic, hydroiodic, nitric, sulfuric, and phosphoric. Organic acids include, but are not limited to, aliphatic, aromatic, carboxylic, and sulfonic organic acids including, but not limited to, formic, acetic, propionic, succinic, benzoic camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, alginic, and galacturonic acid.

As used herein, a composition that is “substantially free” of a compound means that the composition contains less than about 20% by weight, more preferably less than about 10% by weight, even more preferably less than about 5% by weight, and most preferably less than about 3% by weight of the compound.

As used herein, the terms “optically pure,” “enantiomerically pure,” “pure enantiomer,” and “optically pure enantiomer” mean a composition that comprises one enantiomer of a compound and is substantially free of the opposite enantiomer of the compound. A typical optically pure compound comprises greater than about 80% by weight of one enantiomer of the compound and less than about 20% by weight of the opposite enantiomer of the compound, more preferably greater than about 90% by weight of one enantiomer of the compound and less than about 10% by weight of the opposite enantiomer of the compound, even more preferably greater than about 95% by weight of one enantiomer of the compound and less than about 5% by weight of the opposite enantiomer of the compound, and most preferably greater than about 97% by weight of one enantiomer of the compound and less than about 3% by weight of the opposite enantiomer of the compound. For example, optically pure (−) sibutramine comprises at least about 80% by weight (−) sibutramine and less than about 20% by weight (+) sibutramine.

It should be noted that names used herein to identify compounds of the invention may differ from those that are concordant with International Union of Pure and Applied Chemistry (IUPAC) naming conventions. If there is a discrepancy between a structure depicted herein and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4. DETAILED DESCRIPTION OF THE INVENTION

This invention relates, in part, to methods of treating and preventing disorders and conditions in patients that include, but are not limited to: eating disorders such as weight gain and obesity; platelet adhesion; apnea; obsessive-compulsive disorders; affective disorders (e.g., ADHD), depression, or anxiety; male and female sexual function disorders, such as erectile dysfunction; restless leg syndrome; osteoarthritis; irritable bowel syndrome; substance abuse including, nicotine addiction from cigarette smoking or chewing tobacco, and cocaine addiction; migraines; chronic pain; pain, such as neuropathic pain, such as diabetic neuropathy; cerebral function disorders; chronic disorders; and incontinence.

Some methods of the invention comprise administering to a patient in need of treatment or prevention a therapeutically or prophylactically effective amount of racemic sibutramine, or a pharmaceutically acceptable salt, solvate, hydrate, clathrate, or prodrug thereof, in combination with a phosphodiesterase inhibitor.

Other methods of the invention comprise administering to a patient in need of treatment or prevention a therapeutically or prophylactically effective amount of optically pure (−) sibutramine, or a pharmaceutically acceptable salt, solvate, hydrate, clathrate, or prodrug thereof, optionally in combination with a phosphodiesterase inhibitor.

Preferred methods of the invention that comprise the administration of a therapeutically or prophylactically effective amount of (−) sibutramine, or a pharmaceutically acceptable salt, solvate, hydrate, clathrate, or prodrug thereof, avoid adverse effects associated with racemic sibutramine.

As used herein, the term “avoid adverse effects” means to incur fewer or none of the adverse effects of the drug referred to, or to incur at least one of those effects to a lesser degree. Thus, a method that avoids adverse effects associated with racemic sibutramine is a method that incurs fewer of the adverse effects of racemic sibutramine, or that incurs at least one of those adverse effects to a lesser degree.

A first embodiment of the invention encompasses a method of treating or preventing a sexual function disorder in a patient in need of such treatment or prevention, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

Another method of this embodiment is a method of treating or preventing a sexual function disorder in a patient in need of such treatment or prevention, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a therapeutically or prophylactically effective amount of a phosphodiesterase inhibitor.

In a preferred method of this embodiment, optically pure (−) sibutramine, or a pharmaceutically acceptable salt, solvate, hydrate, clathrate, or prodrug thereof, is administered to a patient orally, transdermally, or mucosally.

In another preferred method of this embodiment, the patient in need of treatment or prevention is elderly or postmenstrual.

As used herein, the terms “sexual dysfunction” and “sexual function disorder” encompass sexual dysfunction in men and women caused by psychological and/or physiological factors. Examples of sexual dysfunction include, but are not limited to, erectile dysfunction, vaginal dryness, lack of sexual excitement, or inability to obtain orgasm. The term “sexual dysfunction” further encompasses psycho-sexual dysfunction. Examples of psycho-sexual dysfunction include, but are not limited to, inhibited sexual desire, inhibited sexual excitement, inhibited female orgasm, inhibited male orgasm, premature ejaculation, functional dysparcunia, functional vaginismus, and atypical psychosexual dysfunction.

Another embodiment of the invention encompasses a method of treating or preventing an affective disorder in a patient, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

Another method of this embodiment is a method of treating or preventing an affective disorder in a patient, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a therapeutically or prophylactically effective amount of a phosphodiesterase inhibitor.

Affective disorders include, but are not limited to, depression (e.g., melancholia), attention deficit disorder (including attention deficit disorder with hyperactivity and attention deficit/hyperactivity disorder), bipolar and manic conditions, dysthymic disorder, and cyclothymic disorder. As used herein, the terms “attention deficit disorder” (ADD), “attention deficit disorder with hyperactivity” (ADDH), and “attention deficit/hyperactivity disorder” (AD/HD), are used in accordance with their accepted meanings in the art. See, e.g., Diagnostic and Statistical Manual of Mental Disorders, Fourth Ed., American Psychiatric Association, 1997 (DSM-IV™) and Diagnostic and Statistical Manual of Mental Disorders, 3^{rd} Ed., American Psychiatric Association (1981) (DSM-III™).

A preferred method of this embodiment is a method of treating or preventing attention deficit disorder in children (e.g., ages 3-18). Another preferred method of this embodiment is a method of treating or preventing depression.

As used herein, the term “treating or preventing depression” means relief from or prevention of the symptoms of depression which include, but are not limited to, changes in mood, feelings of intense sadness, despair, mental slowing, loss of concentration, pessimistic worry, agitation, and self-deprecation. Physical changes can also be relieved or prevented by this method, and include, but are not limited to, insomnia, anorexia, decreased energy and libido, and abnormal hormonal circadian rhythms.

Another embodiment of the invention encompasses a method of treating or preventing weight gain or obesity in a patient, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a lipase inhibitor.

As used herein, the term “treating or preventing weight gain or obesity” means reduction of weight, relief from being overweight, treating weight gain caused by the administration of other drugs, relief from gaining weight, or relief from obesity, and prevention from gaining weight, all of which are usually due to unnecessary consumption of food. The invention also encompasses methods of treating or preventing conditions incidental to obesity including, but not limited to, hypertension, such as pulmonary hypertension; cancers, such as breast, colon, gall bladder, and endometrial; gall stones; cardiovascular disease, such as dyslipidemia and carotid intimal medial thickening; hiatial hernia; osteoarthritis; gout; thyroid disease, such as diabetes; gastro-esophogeal reflux disease; menstrual dysfunction; and infertility.

Another embodiment encompasses a method of treating or preventing a disorder associated with the administration of a lipase inhibitor for obesity or weight management, such as, for example, orlistat (XENICAL®), which comprises administering to a patient in need of such treatment or prevention a therapeutically or prophylactically effective amount of racemic sibutramine or optically pure (−) sibutramine, or a pharmaceutically acceptable salt, solvate, hydrate, clathrate, or prodrug thereof. As used herein, the term “treating or preventing a disorder associated with the administration of a lipase inhibitor” means alleviating or reducing adverse effects associated with administration of a lipase inhibitor, which include, but are not limited to, infectious diarrhea, oily fecal spotting, flatus with discharge, fecal urgency, fatty/oily stool, oily evacuation, increased defecation, anal leakage, and fecal incontinence.

Another embodiment of the invention encompasses a method of treating or preventing cerebral function disorder, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

Another method of this embodiment is a method of treating or preventing a cerebral function disorder in a patient, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a therapeutically or prophylactically effective amount of a phosphodiesterase inhibitor.

Cerebral function disorders include, but are not limited to, senile dementia, Alzheimer's type dementia, memory loss, amnesia/amnestic syndrome, disturbance of consciousness, coma, lowering of attention, speech disorders, Parkinson's disease, Lennox syndrome, autism, epilepsy, hyperkinetic syndrome, and schizophrenia. Cerebral function disorders can be induced by factors including, but not limited to, cerebrovascular diseases, such as cerebral infarction, cerebral bleeding, cerebral arteriosclerosis, cerebral venous thrombosis, and head injuries, and conditions having symptoms selected from the group consisting of disturbances of consciousness, senile dementia, coma, lowering of attention, and speech disorders. As used herein, the term “treating or preventing a cerebral function disorder” means relief from or prevention of one or more symptoms associated with cerebral function disorders.

Another embodiment encompasses a method of treating or preventing restless leg syndrome, which comprises administering to a patient in need of such treatment or prevention a therapeutically or prophylactically effective amount of racemic sibutramine or optically pure (−) sibutramine, or a pharmaceutically acceptable salt, solvate, hydrate, clathrate, or prodrug thereof. In a particular method of this embodiment, the racemic or optically pure sibutramine is administered in combination with a phosphodiesterase inhibitor.

In a preferred embodiment, the patient is at least about 50, 60, or 70 years of age. In another preferred method of this embodiment, the racemic or optically pure sibutramine is administered in combination with at least one of pergolide, carbidopa, levodopa, oxycodone, carbamazepine, gabapentin, or pharmaceutically acceptable salts, solvates, hydrates, clathrates, prodrugs, optically and pharmacologically active stereoisomers, or pharmacologically active metabolites thereof.

As used herein, the term “restless leg syndrome” encompasses a disorder that typically occurs during sleep or rest, or just before sleep or rest, and which is characterized by uncomfortable sensations in the legs. The disorder often occurs in patients older than about 50 years of age. Examples of uncomfortable sensations in the legs include, but are not limited to, pulling, drawing, crawling, wormy, boring, tingling, pins and needles, prickly and sometimes painful sensations that are usually accompanied by an overwhelming urge to move the legs. As used herein, the term “restless leg syndrome” also encompasses Ekbom Syndrome, Wittmaack-Ecbom Syndrome, Hereditary Acromelalgia, and Anxieties Tibialis.

Another embodiment of the invention encompasses a method of treating or preventing pain in a patient, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

Another method of this embodiment is a method of treating or preventing pain in a patient, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a therapeutically or prophylactically effective amount of a phosphodiesterase inhibitor.

Another method of this embodiment is a method of treating or preventing an obsessive-compulsive disorder in a patient in need of such treatment or prevention, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

As used herein, the terms “obsessive-compulsive disorder,” “pre-menstrual syndrome,” “anxiety,” and “eating disorder” are used consistently with their accepted meanings in the art. See, e.g., DSM-IV™ and DSM-III™. The term “methods of treating or preventing” when used in connection with these disorders means the amelioration, prevention, or relief from symptoms and/or effects associated with these disorders.

Another embodiment encompasses a method of treating or preventing substance abuse which comprises administering to a patient in need of such treatment or prevention a therapeutically or prophylactically effective amount of optically pure (−) sibutramine, or a pharmaceutically acceptable salt, solvate, hydrate, clathrate, or prodrug thereof. In a particular embodiment, the substance abuse is cocaine addiction or alcohol addiction.

As used herein, the term “substance abuse” encompasses the abuse of, and physical and/or psychological addiction to, drugs or alcohol. The term “substance abuse” further encompasses its accepted meaning in the art. See, e.g., DSM-IV™ and DSM-III™. A preferred method encompassed by this embodiment is a method of treating or preventing cocaine and/or heroin abuse.

Another embodiment encompasses a method of treating or preventing nicotine addiction which comprises administering to a patient in need of such treatment or prevention a therapeutically or prophylactically effective amount of optically pure (−) sibutramine, or a pharmaceutically acceptable salt, solvate, hydrate, clathrate, or prodrug thereof. Nicotine addiction includes nicotine addiction of all known forms, such as addiction to cigarettes, cigars and/or pipes, and chewing tobacco.

Another embodiment encompasses a method of eliciting smoking cessation which comprises administering to a patient who smokes tobacco a therapeutically effective amount of optically pure (−) sibutramine, or a pharmaceutically acceptable salt, solvate, hydrate, clathrate, or prodrug thereof. In a preferred method encompassed by this embodiment, optically pure (−) sibutramine, or pharmaceutically acceptable salt, solvate, hydrate, clathrate, or prodrug thereof, is administered orally, mucosally, or transdermally. In a more preferred method, optically pure (−) sibutramine or pharmaceutically acceptable salt, solvate, hydrate, or clathrate thereof is administered transderrnally.

In another preferred method of this embodiment, optically pure (−) sibutramine, or pharmaceutically acceptable salt, solvate, hydrate, clathrate, or prodrug thereof, is administered in combination with a therapeutically or prophylactically effective amount of nicotine. Preferably, the nicotine and/or optically pure (−) sibutramine or pharmaceutically acceptable salt, solvate, hydrate, clathrate, or prodrug thereof is administered orally, mucosally, or transdermally. More preferably, the nicotine and/or optically pure (−) sibutramine or pharmaceutically acceptable salt, solvate, ester, clathrate, or prodrug thereof is administered transdermally.

Another method encompassed by this embodiment is a method of treating or preventing weight gain associated with smoking cessation which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

Another embodiment encompasses a method of treating or preventing weight gain associated with the administration of other drugs that may induce weight gain, which comprises administering to a patient in need of such treatment or prevention a therapeutically or prophylactically effective amount of optically pure (−) sibutramine, or a pharmaceutically acceptable salt, solvate, ester, clathrate, or prodrug thereof.

Another embodiment encompasses a method of treating or preventing a chronic disorder including, but not limited to, narcolepsy, chronic fatigue syndrome, seasonal affective disorder, fibromyalgia, and premenstrual syndrome (or premenstrual dysphoric disorder), which comprises administering to a patient in need of such treatment or prevention a therapeutically or prophylactically effective amount of optically pure (−) sibutramine, or a pharmaceutically acceptable salt, solvate, hydrate, clathrate, or prodrug thereof. Preferred methods are methods of treating or preventing narcolepsy, premenstrual syndrome, or chronic fatigue syndrome.

Another embodiment encompasses a method of treating or preventing anxiety which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

Another embodiment encompasses a method of treating or preventing an eating disorder including, but not limited to, anorexia, bulimia, binging, and snacking, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

Another embodiment of the invention encompasses a method of treating or preventing migranes in a patient in need of such treatment or prevention, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

Another method of this embodiment is a method of treating or preventing migranes in a patient in need of such treatment or prevention, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a therapeutically or prophylactically effective amount of a phosphodiesterase inhibitor.

Another embodiment encompasses a method of treating or preventing incontinence which comprises administering to a patient in need of such treatment or prevention a therapeutically or prophylactically effective amount of a optically pure (−) sibutramine, or a pharmaceutically acceptable salt, solvate, ester, clathrate, or prodrug thereof. In particular, optically pure (−) sibutramine can be used to treat fecal incontinence, stress urinary incontinence (“SUI”), urinary exertional incontinence, urge incontinence, reflex incontinence, passive incontinence, anal leakage, and overflow incontinence.

As used herein, the term “treating or preventing incontinence” means treatment, prevention of, or relief from the symptoms of incontinence including involuntary voiding of feces or urine, and dribbling or leakage or feces or urine, which may be due to one or more causes including, but not limited to, pathology altering sphincter control, loss of cognitive function, overdistention of the bladder, hyper-reflexia and/or involuntary urethral relaxation, weakness of the muscles associated with the bladder or neurologic abnormalities.

A preferred method encompassed by this embodiment is a method of treating or preventing stress urinary incontinence. In a further preferred method encompassed by this embodiment, the patient is an elder human of an age greater than about 50 or a child of an age less than about 13.

In a specific embodiment of each of the methods of the invention, a therapeutically or prophylactically effective amount of optically pure (−) sibutramine is administered to a patient in combination with an additional pharmacologically active compound. Examples of additional pharmacologically active compounds include, but are not limited to, phosphodiesterase inhibitors and lipase inhibitors. As discussed in more detail herein, the particular additional pharmacologically active compound used in a method will depend upon the disease or condition being treated or prevented, as well as the particular patient being treated.

The invention also encompasses pharmaceutical compositions and single unit dosage forms that can be used, for example, in the methods described herein. One embodiment of the invention encompasses a pharmaceutical composition or dosage form that comprises optically pure (−) sibutramine, or a pharmaceutically acceptable salt, solvate, hydrate, clathrate, or prodrug thereof. Other pharmaceutical compositions and single unit dosage forms of the invention comprise racemic sibutramine or optically pure (−) sibutramine, or a pharmaceutically acceptable salt, solvate, hydrate, clathrate, or prodrug thereof, and an additional pharmacologically active compound.

4.1. Preparation of (−) Sibutramine

The optically purified stereoisomers of sibutramine are most readily obtained by resolving the racemic mixture of sibutramine prepared by following the synthetic procedures disclosed herein or those described in U.S. Pat. Nos. 4,522,828 and 4,476,680, the disclosures of which are hereby incorporated by reference. A preferred technique is resolution by fractional crystallization of diastereomeric salts formed with optically active resolving agents. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972).

Since sibutramine is a basic amine, diastereomeric salts suitable for separation by fractional crystallization are readily formed by addition of chiral acid resolving agents in optically pure form. Suitable resolving agents for use herein include optically pure tartaric acid and its derivatives, camphorsulfonic acid, mandelic acid and derivatives thereof, and other optically active acids. The desired (−) sibutramine isomer may be recovered either from the crystallized diastereomer or from the mother liquor, depending on the solubility properties of the particular acid resolving agent employed and depending on the particular acid enantiomer used. The identity and optical purity of the particular sibutramine isomer so recovered may be determined by polarimetry or other analytical methods.

4.2. Methods of Treatment and Prevention

In each of the methods of the invention, a therapeutically or prophylactically effective amount of racemic sibutramine or optically pure (−) sibutramine, or a pharmaceutically acceptable salt, solvate, hydrate, clathrate, or prodrug thereof, is administered to a patient.

The magnitude of a prophylactic or therapeutic dose of racemic sibutramine or optically pure (−) sibutramine in the acute or chronic management of disease will vary with the severity of the condition to be treated and the route of administration. The dose and perhaps the dose frequency will also vary according to age, body weight, response, and the past medical history of the individual patient. In general, the recommended daily dose range for the conditions described herein lie within the range of from about 1 mg to about 60 mg per day, given as a single once-a-day dose in the morning or as divided doses throughout the day. Preferably, a daily dose range should be from about 2 mg to about 50 mg per day; and most preferably, a daily dose range should be between about 5 mg and about 30 mg per day. In managing the patient, the therapy should be initiated at a lower dose, perhaps about 5 to about 15 mg, and increased if necessary up to about 5 mg per day as either a single dose or divided doses, depending on the patient's global response. It is further recommended that patients aged over 65 years should receive doses in the range of about 5 to about 30 mg per day depending on global response. It may be necessary to use dosages outside these ranges.

Optionally, racemic sibutramine or optically pure (−) sibutramine is adjunctively administered (i.e., administered in combination) with one or more additional pharmacologically active compounds. For example, optically pure (−) sibutramine and an additional pharmacologically active compound can be administered to a patient as a combination, concurrently but separately, or sequentially by any suitable route. Suitable routes of administration include oral, mucosal (e.g., nasal, sublingual, buccal, rectal, and vaginal), parenteral (e.g., intravenous, intramuscular or subcutaneous), and transdermal routes.

As physicians and those skilled in the art of pharmacology will readily appreciate, the particular additional pharmacologically active compounds that can be administered in combination with a optically pure (−) sibutramine will depend on the particular disease or condition being treated or prevented, and may also depend on the age and health of the patient to which the compounds are to be administered.

Additional pharmacologically active compounds that can be used in the methods and compositions of the invention include, but are not limited to, phosphodiesterase and lipase inhibitors. Examples of phosphodiesterase inhibitors that can be used in compositions and methods of the invention include, but are not limited to, those disclosed in U.S. Pat. No. 5,250,534; U.S. Pat. No. 5,719,283; U.S. Pat. No. 6,127,363; WO 94/28902; WO 97/03675; WO 98/06722, all of which are expressly incorporated herein by reference in their entirety. Preferred phosphodiesterase inhibitors are PDE5 and PDE6 inhibitors. Particular phosphodiesterase inhibitors include, but are not limited to, sildenophil (Viagra®), desmethylsildenophil, vinopocetine, milrinone, amrinone, pimobendan, cilostamide, enoximone, peroximone, vesnarinone, rolipran, R020-1724, zaprinast, dipyridamole, and pharmaceutically acceptable salts, solvates, hydrates, clathrates, prodrugs, optically and pharmacologically active stereoisomers, and pharmacologically active metabolites thereof.

Suitable daily dosage ranges of additional pharmacologically active compounds that can be adjunctively administered with racemic sibutramine or optically pure (−) sibutramine can be readily determined by those skilled in the art following dosages reported in the literature and recommended in the Physician 's Desk Reference®.

For example, suitable daily dosage ranges of phosphodiesterase inhibitors can be readily determined by those skilled in the art. In general, the total daily dose of a phosphodiesterase inhibitor will be from about 0.5 mg to about 500 mg, from about 1 mg to about 350 mg, or from about 2 mg to about 250 mg.

The dosage amounts and frequencies provided herein are encompassed by the terms “therapeutically effective,” “prophylactically effective,” and “therapeutically or prophylactically effective” as used herein. When used in connection with an amount of optically pure (-) sibutramine, these terms further encompass an amount of optically pure (−) sibutramine that induces fewer or less sever adverse effects than are associated with the administration of racemic sibutramine. Adverse effects associated with racemic sibutramine include, but are not limited to, significant increases in supine and standing heart rate, including tachycardia, increased blood pressure (hypertension), increased psychomotor activity, dry mouth, dental caries, constipation, hypohidrosis, blurred or blurry vision, tension, mydriasis, seizures, formation of gallstones, renal/hepatic dysfunction, fevers, arthritis, agitation, leg cramps, hypertonia, abnormal thinking, bronchitis, dyspnea, pruritus, amblyopia, menstrual disorder, ecchymosis/bleeding disorders, interstitial nephritis, and nervousness. However, the induction of fewer or less severe adverse-effects is attributable to the administration of a sibutramine metabolite and the efficacy of which may be less apparent or absent with the administration of a combination therapy.

4.3. Pharmaceutical Compositions and Dosage Forms

Pharmaceutical compositions and dosage forms of the invention comprise one or more of the active ingredients disclosed herein (e.g., (−) sibutramine, or a pharmaceutically acceptable prodrug, salt, solvate, hydrate, or clathrate thereof). Pharmaceutical compositions and dosage forms of the invention typically also comprise one or more pharmaceutically acceptable excipients or diluents.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of sexual dysfunction or a related disorder may contain larger amounts of one or more of the active ingredients it comprises than a dosage form used in the chronic treatment of sexual dysfunction. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington 's Pharmaceutical Sciences, 18th ed., Mack Publishing, Easton PA (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise optically pure (−) sibutramine, or a pharmaceutically acceptable salt, solvate, clathrate, hydrate, or prodrug thereof in an amount of from about 1 mg to about 60 mg, preferably in an amount of from about 3 mg to about 50 mg, more preferably in an amount of from about 5 mg to about 30 mg, and most preferably in an amount of from about 10 mg to about 25 mg.

4.3.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton PA (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating vagents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103® and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

4.3.2. Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds. 4.3.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

4.3.4. Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.3.5. Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a unit dosage form of racemic sibutramine or optically pure (−) sibutramine, or a pharmaceutically acceptable prodrug, salt, solvate, hydrate, or clathrate thereof, and a unit dosage form of a second active ingredient. Examples of second active ingredients include, but are not limited to, phosphodiesterase inhibitors and lipase inhibitors.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

### 5.1. Example 1

Synthesis and Optical Resolution of Sibutramine

Synthesis of 1-(4-Chlorophenvl)cyclobutanecarbonitrile

To a suspension of NaH (17.6 g 60%, washed with hexane) in dimethylsulfoxide (150 mL) at room temperature with mechanical stirring was added over a one hour period a mixture of chlorbenzylnitrile (30.3 g) and 1,3-dibromopropane (22.3 mL, 44.5 g). The reaction mixture was stirred for an additional 1 hour, and isopropyl alcohol (10 mL) was added slowly to quench excess NaH. Water (150 mL) was added. The reaction mixture was extracted with t-butyl methyl ether (MTBE) (2×200 mL), and the combined extracts were washed with water (3×200 mL), brine, and dried over MgSO₄. The solvent was removed in a rotoevaporator, and the final product was purified by distillation to give the title compound (22 g, 56%) as pale yellow oil, bp 110-120° C./1.0 mm Hg. The product was characterized by ¹H NMR.

Synthesis of 1-[l -(4-chlorophenyl)cyclobutyl]-3-methylbutylamine

A solution of isobutylmagnesium bromide (2M, 108 mL) in diethyl ether (Aldrich) was concentrated to remove most of the ether. The residue was dissolved in toluene (150 mL), followed by addition of the nitrile made above (22 g). The reaction mixture was heated to 105° C. for 17 hours. The reaction mixture was cooled to room temperature, and added to a slurry of NaBH₄ in isopropyl alcohol (450 mL). The reaction mixture was heated under reflux for 6 hours, cooled to room temperature and concentrated. The residue was diluted with water (350 mL), and extracted with ethyl acetate (3×200 mL). The combined extracts were washed with water (100 mL), and dried (MgSO₄), and concentrated to give 24.2 g crude product (83%).

Synthesis of Sibutramine Free Base

1-[1-(4-chlorophenyl)cyclobutyl]-3-methylbutylamine (21.6 g) was added to formic acid (27 mL) and aqueous formaldehyde (46 mL). The reaction mixture was heated to 85-95° C. for 18 hours and was cooled to room temperature. 30% NaOH was added until the mixture was basic (pH >11). The solution was extracted with chloroform (3×200 mL) and the extracts were combined and washed with water and brine and concentrated to give 15 g product.

Sibutramine HCl

Sibutramine free base (2.25 g) was dissolved in MTBE (20 mL) and that solution was added to 20 mL 1M HCl in diethyl ether. The reaction mixture was stirred for 30 minutes, and the solid was collected by filtration to give 1.73 g after drying. The product was characterized by ¹H NMR.

Resolution of Sibutramine

12.3 g racemic sibutramine was dissolved in ethyl acetate (85 mL), and a solution of 21.7 g L-dibenzyltartaric acid (“L-DBTA”) in ethyl acetate (85 mL) was added thereto. The reaction mixture was heated to reflux and cooled to room temperature. The white precipitate was collected (ee of salt is ca 85%). The solid was then suspended in 220 mL ethy acetate and heated at reflux for 30 minutes. The solid was collected to give>95% ee. The salt was further crystallized in isopropyl alcohol (450 mL) to give 11.3 g of salt with>99.3% ee. (−)-Sibutramine L-DBTA (yield 76%). Free base was obtained by treatment of the salt with saturated aqueous NaHCO₃ and extracted with chloroform. The (−)-sibutramine HCl salt was obtained with treatment of the free base with HCl/Et₂O as described above. Optical rotation of the HCl salt was [α]=3.15(c=0.9, H₂O), ¹H NMR ¹³C(CD₃OD), and M⁺=279. The resolution mother liquor was treated with NaOH to give the partially enriched (+)-sibutramine and was then treated with D-DBTA as described above to give (+)-sibutramine-D-DBTA salt with >99.3% ee. The sibutramine enantiomers were characterized by ¹H and ¹³C NMR: M⁺=279. The material was also characterized by HPLC and Chiral HPLC.

### 5.2. Example 2

Pharmacological Study of Racemic and Optically Pure Sibutramine

A pharmacologic study is conducted to determine the relative potency, comparative efficacy, binding affinity, and toxicity of the enantiomers and racemic mixture of sibutramine. The profile of relative specificity of monoamine reuptake inhibition is determined from the compound's inhibition of norepinephrine (NE) (variously known as noradrenaline) reuptake in brain tissue with that of the inhibition of dopamine (DA) and serotonin (5HT) reuptake.

High-affinity uptake of the ³H-radiomonoamines is studied in synaptosomal preparations prepared from rat corpus striatum (for inhibition of DA reuptake) and cerebral cortex (for 5HT and NE) using methods published by Kula et al., Life Sciences 34(26): 2567-2575, 1984, and Baldessarini et al, Life Sciences 39: 1765-1777, 1986. Tissues are freshly dissected on ice and weighed. Following homogenization by hand (14 strokes in 10-35 vols of ice-cold isotonic 0.32M sucrose, containing nilamide, 34 μM) in a Teflon-on-glass homogenizer, the tissue is centrifuged for ten (10) minutes at 900 x g; the supernatant ‘solution’ that results contains synaptosomes that are used without further treatment. Each assay tube contains 50 μL of the cerebral homogenate, radiolabelled-³H-monoamine, and the test compound (e.g., the pure sibutramine enantiomers, the racemate, and appropriate standards) in a freshly prepared physiologic buffer solution with a final volume of 0.5 mL. Tissues are preincubated for 15 minutes at 37° C. before the assay. Tubes are held on ice until the start of incubation which is initiated by adding ³H-amine to provide a final concentration of 0.1 MM. Tubes are incubated at 37° C. for 10 minutes with ³H-DA (26 Ci/mmol) and for 20 minutes with ³H-5HT (about 20 Ci/mmol) and ³H-NE (about 20 Ci/mmol). The specific activity of the radiomonoamine will vary with available material and is not critical. The reaction is terminated by immersion in ice and dilution with 3 ml of ice cold isotonic saline solution containing 20 mM TRIS buffer (pH 7.0). These solutions are filtered through cellulose ester microfilters, followed by washing with two 3 mL volumes of the same buffer. The filter is then counted for ³H-radioactivity in 3.5 mL of Polyfluor at˜50% efficiency for tritiuM. Blanks (either incubated at 0° C. or incubated with specific, known uptake inhibitors of DA [GRB-12909, 10 MM], 5HT-zimelidine 10 μM], or of NE [desipramine 10 μM)) are usually indistinguishable from assays performed without tissue and average 2-3% of total CPM.

Comparison of the amounts of ³H-radioactivity retained on the filters provides an indication of the relative abilities of the pure enantiomers and racemic mixture of sibutramine (and of known DA-, 5HT-, or NE-reuptake inhibitors) to block the reuptake of these monoamines in those tissues. This information is useful in gauging the relative potency and efficacy of racemic sibutramine and its enantiomers.

The acute toxicities of the enantiomers of sibutramine and of the racemic mixture thereof are determined in studies in which rats are administered progressively higher doses (mg/kg) of the pure isomers or racemate. That lethal dose which, when administered orally, causes death of 50% of the test animals, is reported as the LD₅₀. Comparison of LD₅₀ values for the enantiomers and racemate provides a measure of the relative toxicity of the compositions.

### 5.3. Example 2

Oral Formulations

CAPSULES

| | Quantity per | | |
|---|---|---|---|
| | Capsule in mg | | |
| Formula | A | B | C |
| Active ingredient | 10.0 | 20.0 | 30.0 |
| (-) sibutramine | | | |
| Lactose | 70.0 | 60.0 | 95.0 |
| Corn Starch | 19.5 | 19.5 | 24.5 |
| Magnesium Stearate | 0.05 | 0.05 | 0.05 |
| Compression | 100.0 | 100.0 | 100.0 |
| Weight | | | |

The active ingredient, (−) sibutramine, the lactose and corn starch are blended until uniform; then the magnesium stearate is blended into the resulting powder. The resulting mixture is encapsulated into suitably sized two-piece hard gelatin capsules.

TABLETS

| | Quantity per Tablet in mg | | |
|---|---|---|---|
| Formula | A | B | C |
| Active ingredient | 0 | 20 | 30 |
| (-) sibutramine | | | |
| Lactose | 94 | 84 | 74 |
| Starch BP | 30 | 30 | 30 |
| Pregelantinized Maize Starch | 15 | 15 | 15 |
| Magnesium Stearate | 1 | 1 | 1 |
| Compression Weight | 150 | 150 | 150 |

The active ingredients sieved through a suitable sieve and blended with lactose, starch, and pregelatinized maize starch. Suitable volumes of purified water are added, and the powders are granulated. After drying, the granules are screened and blended with the magnesium stearate. The granules are then compressed into tablets using punches.

Tables of other strengths may be prepared by altering the ratio of active ingredient to lactose or to the compression weight and using punches to suit.

What is claimed is:

1. A method of treating or preventing a sexual function disorder in a patient, which comprises administering to a patient in need of such treatment or prevention therapeutically or prophylactically effective amounts of optically pure (−) sibutramine, or a pharmaceutically acceptable salt, solvate, hydrate, clathrate, or prodrug thereof, and a phosphodiesterase inhibitor.

2. A method of treating or preventing an affective disorder in a patient in need of such treatment or prevention, which comprises administering to a patient in need of such treatment or prevention therapeutically or prophylactically effective amounts of optically pure (−) sibutramine, or a pharmaceutically acceptable salt, solvate, hydrate, clathrate, or prodrug thereof, and a phosphodiesterase inhibitor.

3. The method of claim 2 wherein the affective disorder is attention deficit disorder, depression, or anxiety.

4. A method of treating or preventing weight gain or obesity in a patient, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof, optionally in combination with a lipase inhibitor.

5. A method of treating or preventing a disorder associated with the administration of a lipase inhibitor for obesity or weight management, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

6. A method of treating or preventing cerebral function disorder in a patient, which comprises administering to a patient in need of such treatment or prevention therapeutically or prophylactically effective amounts of racemic sibutramine, or a pharmaceutically acceptable salt, solvate, hydrate, clathrate, or prodrug thereof, and a phosphodiesterase inhibitor.

7. The method of claim 6 wherein the cerebral function disorder is senile dementia, Alzheimer's type dementia, memory loss, amnesia/amnestic syndrome, disturbance of consciousness, coma, lowering of attention, speech disorders, Parkinson's disease, Lennox syndrome, autism, epilepsy, hyperkinetic syndrome, or schizophrenia.

8. A method of treating or preventing restless leg syndrome, which comprises administering to a patient in need of such treatment or prevention a therapeutically or prophylactically effective amount of optically pure (−) sibutramine, or a pharmaceutically acceptable salt, solvate, hydrate, clathrate, or prodrug thereof.

9. The method of claim 8 which further comprises the administration of pergolide, carbidopa, levodopa, oxycodone, carbamazepine, or gabapentin, or a pharmaceutically acceptable salt, solvate, hydrate, clathrate, prodrug, optically and pharmacologically active stereoisomer, or pharmacologically active metabolite thereof.

10. A method of treating or preventing pain in a patient, which comprises administering to a patient in need of such treatment or prevention therapeutically or prophylactically effective amounts of optically pure (-) sibutramine, or a pharmaceutically acceptable salt, solvate, hydrate, clathrate, or prodrug thereof, and a phosphodiesterase inhibitor.

11. A method of treating or preventing a migrane in a patient, which comprises administering to a patient in need of such treatment or prevention therapeutically or prophylactically effective amounts of optically pure (−) sibutramine, or a pharmaceutically acceptable salt, solvate, hydrate, clathrate, or prodrug thereof, and a phosphodiesterase inhibitor.

12. The method of claim 1, 2, 4, 6, 8, 10, or 11 wherein the therapeutically or prophylactically effective amount of optically pure (−) sibutramine is from about 1 mg to about 60 mg per day.

13. The method of claim 12 wherein the therapeutically or prophylactically effective amount of optically pure (−) sibutramine is from about 2 mg to about 50 mg per day.

14. The method of claim 13 wherein the therapeutically or prophylactically effective amount of optically pure (−) sibutramine is from about 5 mg to about 30 mg per day.

15. The method of claim 1, 2, 6, 10, or 11 wherein the phosphodiesterase inhibitor is sildenophil, desmethylsildenophil, vinopocetine, milrinone, amrinone, pimobendan, cilostamide, enoximone, peroximone, vesnannone, rolipram, R020-1724, zaprinast, dipyridamole, or a pharmaceutically acceptable salt, solvate, hydrate, clathrate, prodrug, optically and pharmacologically active stereoisomer, or a pharmacologically active metabolite thereof.

16. A pharmaceutical composition comprising optically pure (−) sibutramine, or a pharmaceutically acceptable salt, solvate, hydrate, clathrate, or prodrug thereof, and a phosphodiesterase inhibitor.

17. The pharmaceutical composition of claim 16 wherein the phosphodiesterase inhibitor is sildenophil, desmethylsildenophil, vinopocetine, milrinone, amrinone, pimobendan, cilostamide, enoximone, peroximone, vesnarinone, rolipram, R020-1724, zaprinast, dipyridamole, or a pharmaceutically acceptable salt, solvate, hydrate, clathrate, prodrug, optically and pharmacologically active stereoisomer, or a pharmacologically active metabolite thereof.

18. The pharmaceutical composition of claim 16 wherein the optically pure (−) sibutramine is in an amount of from about 1 mg to about 60 mg.

19. The pharmaceutical composition of claim 18 wherein the optically pure (−) sibutramine is in an amount of from about 2 mg to about 50 mg.

20. The pharmaceutical composition of claim 19 wherein the optically pure (−) sibutramine is in an amount of from about 5 mg to about 30 mg.

21. The pharmaceutical composition of claim 16 wherein the phosphodiesterase inhibitor is in an amount of from about 0.5 mg to about 500 mg.

22. The pharmaceutical composition of claim 21 wherein the phosphodiesterase inhibitor is in an amount of from about 1 mg to about 350 mg.

23. The pharmaceutical composition of claim 22 wherein the phosphodiesterase inhibitor is in an amount of from about 2 mg to about 250 mg.

24. The pharmaceutical composition of claim 16 wherein the pharmaceutical composition is adapted for oral, mucosal, rectal, parenteral, transdermal, or subcutaneous administration.

25. The pharmaceutical composition of claim 24 wherein the pharmaceutical composition is adapted for oral, mucosal, or transdermal administration.
